(19) United States
(12) Patent Application Publication
(10) Pub. No.: US 2007/0081967 A1
(43) Pub. Date: Apr. 12, 2007

(54) Revitalizing hair follicles with total parenteral nutrition or hyperalimentation for maximum hair growth

(76) Inventors: John Cerullo, Flushing, NY (US); Willard Louey, Elmhurst, NY (US)

Correspondence Address: Jeong Han Oh, 1062 Westmoreland Avenue, Syracuse, NY, 13210, US

(21) Appl. No.: 11/248,580

(22) Filed: Oct. 12, 2005

(51) Int. Cl. A61K 8/97; A61K 36/889; A61K 31/7048; A61K 31/455; A61K 31/353

(52) U.S. Cl. 424/74; 424/727; 514/27; 514/356; 514/456; 514/574

(57) ABSTRACT

The present invention relates to composition and method for revitalizing hair growth, rebuilding and restoring the natural integrity of the hair follicles wherein a first treatment formulation containing active ingredients comprising of vinpocetine, niacin, proanthocyanidins, azelaic acid, isopropyl alcohol, propylene glycol, and water is applied to the scalp as a liquid suspension, lotion, gel or cream, following, a second treatment formulation containing active ingredients comprising of total parenteral nutrition or hyperalimentation, folic acid, propylene glycol, and water is applied to the scalp as a liquid suspension, lotion, gel, or cream, and lastly, a third treatment formulation for men containing sal palmetto, niacin, vinpocetine, folic acid, pantothenic acid, Vitamin A, Vitamin B-6, Zinc, PABA, grape seed extract, thiamine, and biotin, and a third treatment formulation for women comprising of niacin, vitamin A, thiamine, vitamin B-6, pantothenic acid, biotin, PABA, zinc, vitamin e, citrus bioflavonoids, calcium, riboflavin, and inositol.

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### References Cited [Referenced By]

U.S. Patent Documents

| | | | |
|---|---|---|---|
| 5972345 | October, 1999 | Chizick, et al. | 424/727 |
| 6013279 | January, 2000 | Klett-Loch | 424/451 |
| 5750108 | May, 1998 | Edwards | 424/727 |
| 6045801 | April, 2000 | Miyauchi, et al. | 424/744 |
| 5512275 | April, 1996 | Buck | 424/70.1 |
| 6596266 | July, 2003 | Catalfo, et al. | 424/74 |
| 6103273 | August, 2000 | Antoun | 424/642 |
| 6187815 | February, 2001 | Hallam, et al. | 514/535 |
| 6019976 | February, 2000 | Bryant | 424/727 |
| 6358541 | March, 2002 | Goodman | 424/727 |
| Pat App | April, 2004 | Gupta | 424/401 |
| 20040081672 | | | |

### OTHER REFERENCES

“Procyanidin oligomers selectively and intensively promote proliferation of mouse hair epithelial cells in vitro and activate hair follicle growth in vivo,” J. Invest. Dermatology, 1999, 112 (3):310-6.
Harrap, G., Dolphin, S., and Albiston, L., “The effect of age on hair root amino acid levels in human subjects,” Unilever Research Port Sunlight, Bebington, Wirral, UK.
Miyazaki, M. “The effect of a cerebral vasodilator, vinpocetine, on cerebral vascular resistance evaluated by the Doppler ultrasonic technique in patients with cerebrovascular disease,” Angiology, 1995 January; 46(1): 53-8.
Gulyas, B. et al., “Cerebral uptake of vinpocetine and ethanol in cynomolgous monkeys: a comparative preclinical PET study,” Nuclear Medicine and Biology, 2002; 29: 753-759.
Akama, H. et al., “Crystal Structure of Minoxidil,” Analytical Sciences, 2004, Vol. 20 .

### BACKGROUND OF THE INVENTION

1. Field of the Invention

This invention is directed to rebuilding and repairing of the hair follicles, stimulation of hair growth, and revitalization of the hair growth cycles using the synergistic effect of hyperalimentation, vinpocetine, niacin, proanthocyanidins, azelaic acid, and combination of vitamins.

2. Background of the Invention

The hair follicle is one of the few human tissues containing stem cells. The stem cells are interspersed within the basal layer of the outer root sheath and in an area called the bulge. From this reservoir stem cells migrate to hair matrix and start to divide and differentiate. Their behavior is controlled by numerous cytokines produced by cells of the dermal papilla. Dermal papilla cells and some cells of the inner and outer sheaths of the follicle from androgen-dependent hairs have androgen receptors in their cytoplasm and nucleus. Androgen indirectly controls hair growth by influencing the synthesis and release of cytokines from the dermal papilla cells. Drugs affecting hair growth belong to one of the following groups: Cytotoxic drugs, antiandrogens, and drugs acting on potassium channels. Further development of drug selective for certain steps in the process of hair growth will enable more successful therapy of hair growth disorders.

During fetal life the skin is covered with lanugo hairs. Around the eighth month of development this hair is usually shed. A second generation of lanugo hairs then starts growing and lasts until the first three or four months of extrauterine life are completed. After all lanugo hairs have disappeared, two types of hair emerge: vellus and terminal. Vellus hairs are thin, occasionally pigmented and short. All skin is covered with VELLUS hairs with the exception of skin on the palms, soles, volar side of the fingers, penile glands and labia minora et majora (only on the internal side) Under the influence of diverse local and systemic factors vellus hairs are in certain regions transformed to terminal hairs. Terminal hairs are thick, pigmented and medullated.

The portion of hair protruding above the level of the epidermis is called the hair shaft, and the portion within the follicle is the hair root. While terminal hairs are composed of medulla, cortex, and cuticle. Vellus hairs lack a medulla. A few rows of the incompletely keratinized cells form medulla, which is in the middle of the hair shaft. The cortex is built with several rows of completely keratinized fusiform cells; it gives strength to the hair. Cortex is covered with cuticle—one row of flat, keratinized cells arranged like tiles on the roof.

The root of the hair is contained in the follicle. The hair follicle is composed of: epithelial and connective tissue sheaths. The epithelial sheath, which is in close contact with the hair root, has two layers: inner and outer. The inner layer is composed of three sublayers: an inner layer, the cuticle, which is similar and in close contact with the hair cuticle; a middle layer (Huxley's layer) made of a few rows of square cells; and an outer, Henle's layer, made of one row of polygonal, flattened cells. The outer epithelial layer is considered to be a down growth of epidermis, with the spinous layer inside and the basal is thickened and known as the vitreous membrane. A connective tissue sheath is an extension of the dermis—it has two layers, inner papillary and outer reticular.

The bottom of the hair is enlarged and made of cells with high potential for division and differentiation. These cells comprise what is known as the hair matrix. The hair matrix cells divide and move up the follicle, differentiating into either hair cells or inner epithelial sheath cells. Among matrix stem cells there are melanocytes producing pigment of the hair. The pigment is synthesized from the amino acid Tyrosine (catalysed by the enzyme phenoloxydase) and transformed through dopa to dopaquinon. Further transformation of dopaquinon proceeds in two direction—either spontaneous transformation to indolquinon or through the addition of the amino acids cystein. Polymerization of indolquinon only produces the dark pigment, pheomelanin. Matrix cells during their differentiation ingest (by phagocytosis) melanin or pheomelanin from dendritic elongations of melanocytes. This is how hair assumes its color: black if melanin is dominant, and yellow or red if pheomelanin is the major pigment. The portion of connective tissue root sheath that is in intimate contact with the hair matrix is known as the dermal papilla. It has a major role in regulating hair growth.

Hairs grow in cycles which are not synchronized in human beings; each hair enters phases of the growth cycle at a different time. There are three phases of the hair growth cycle: anagen, catagen, and telogen. Anagen is the phase of active hair growth approximately 90% of all hairs are in the anagen phase. They will live for 2 to 6 years, depending on location of the shin region. After anagen is completed, the hair enters catagen. During this short phase (2-3 weeks) the matrix cells gradually stop dividing and eventually keratinize. When full keratinization hairs fall out, and a new matrix is gradually formed from stem cells in basal layer of outer epithelial root sheath bulge. A new hair starts to grow and the follicle returns to anagen phase.

Stem cells of the hair follicle are gathered in the basal layer of the outer root sheath bulge. It is from these cells that matrix cells are formed. Growth and differentiation of the matrix cells are under the influence of substances produced by cells of the dermal papilla. On the other hand, the secretory activity of the dermal papilla is controlled either by substances produced in cells of the spinous layer of the outer root sheath or by hormones. Cells of the spinous layer produces peptides greater than 3000 daltons which increase the number of papilla cell mitoses two or five times. It was recently discovered that basic fibroblast growth factor (bFGF) and platelet-derived growth factor (PDGF) potentiate the growth of dermal papilla cells. It is proposed that these proteins increase the synthesis of Stromelysis (an enzyme, matrix metalloproteinase) which acts on the papilla cells and accelerates their growth. Another cytokine, transforming growth factor beta (TGFB), inhibits Mitogen-Induced dermal papilla cell proliferation. On the other hand, dermal papilla cells produce numerous cytokines which influence proliferation of hair matrix cells. Some of them are stimulators, and some inhibitors. Interleukin 1-Alpha (IL-1Alpha)—inhibits growth of hair and follicle, but only after 2-4 days of latency. The increase of IL-1 alpha concentration in extracellular fluid during inflammation could be one of the reasons for alopecia following certain infectious diseases. Apart from IL-1 alpha, both fibroblast growth factors type 5 (FGF5) is an especially potent inhibitor. Receptors for these ligands were found by immunohistochemical methods on papilla cells, matrix cells and stem cells in the bulge region of the hair follicle. Another cytokine produced by cells of the dermal papilla, keratinocyte growth factor (KGF), induces extensive hair growth in murine models of alopecia. Receptors of KGF were found on keratinocytes in the basal epidermis and throughout developing hair follicles of rat embryos and neonates. Insulin-like growth factor I (IGF-I) also accelerates, in a concentration dependent manner, growth of hair and hair follicles. The actions of IGF_I are modulated by proteins produced in dermal papilla cells which bind IGF (insulin-like growth factors-binding protein: IGFBPs) the exact mechanism of modulation has not yet been resolved. However, it has been shown that IGFBP-3 forms a complex with free IGF-I to reduce the concentration of IGF-I available for stimulation of hair elongation and maintenance of the anagen phase. Insulin itself has the same effect as IGF-I; it has been observed that body hair in patients with hyperinsulinism has a male distribution pattern. On the other hand, growth hormone somatotopin has no direct influence on follicle and hair growth.

Androgens have diverse effects on hair in different body regions. Effects vary from essentially non existent (e.g. on eye-lashes), weak on temporal and suboccipital (region hair), moderate (on extremity hair), or strong (on facial, parietal region, pubic, chest, and axillary hair). Androgens bind to receptors both in the cytoplasm and nuclei of dermal papilla cells and some cells of the sheaths of the follicles, but only if the hair is in anagen of telogen. Two molecular forms of androgen receptors have been proposed: active and inactive. The monomer form has a much greater affinity for androgens. Four monomer molecules aggregate to form a tetramer in a reversible reaction. Necessary factors are glutathione and the enzyme, endogenous disulfide converting factor. The complex of androgen hormone-receptor moves to the cell nucleus and there enables expression of genes coding cytokines. Cells of the dermal papilla synthesize and secrete cytokines which control growth and differentiation, however for hair if the parietal region the cytokines act as inhibitors, leading to follicle atrophy.

Numerous factors affect the number and activity of androgen receptors in dermal papilla cells. Retinoic acid, if used for a long time, may reduce the number of androgen receptors by 30-40 percent. Vitamin B6 reduces by 35-40% the extent of protein synthesis observed after androgen receptor activation. Among all androgens, dermal papilla cells are most affected by 5-alpha-dihydrotestosterone. It is synthesized in these cells from testosterone under catalytic action of the enzyme 5-alpha-reductase. This enzyme exists in two forms—Type I and Type II. 5-alpha-dihydrotestosterone is further reduced to 3-alpha-androstanediol which, after conjugation with glucuronic acid, is excreted in urine. Plasma and urine levels of 3-alpha-androstanediol glucuronide are the most precise clinical indicators of the extent of testosterone transformation to 5-alpha-DHT.

Growth of androgen-dependent hairs can be influenced in several ways: by decreasing androgen production, by blocking testosterone transformation to 5-Alpha-DHT or by blocking androgen receptors. Androgen production can be decreased either surgically (removing of hormone-producing ovarian or adrenal tumor or with drugs. Transformation of testosterone to 5-alpha-DHT can successfully be interrupted with inhibitors of 5-alpha-reductase.

There currently exists many forms of compounds for the purposes of combating androgenetic alopecia and there is still considerable research and development in this field. “Finasteride” described in U.S. Pat. No. 5,981,543, marketed under the name PROSCAR.RTM by Merck is an inhibitor of 5-alpha-reductase, and is used currently as a potent synthetic prescription drug. It has been used to treat urinary problems associated with enlargement of the prostate. Finasteride also is used by some balding men to stimulate hair growth. If hair growth is going to occur with the use of finasteride, it usually occurs after the medicine has been used for about 3 months and lasts only as long as the medicine continues to be used. The new hair will be lost within 1 year after finasteride treatment is stopped. There are significant side effects with finasteride such as erectile dysfunction, decrease in the male libido, decrease in the amount of semen released during sex, and some instances cancer. The success rate for treating alopecia is 40%.

Another widely available compound currently sold as an over-the-counter drug, used by both men and women, is “Minoxidil” described in U.S. Pat. No. 4,596,812 sold under ROGAINE.RTM., a vasodilator and increases the circulation of blood. The original use for Minoxidil was angina and high blood pressure but due to its potentcy caused increase in blood pressure and discontinued as a cardiac drug. Topical Minoxidil has shown to increase hair growth in men and women but also has significant side effects such as systemic absorption of the compound over time and can cause heart problems, lowered blood pressure, dizziness, and dry mouth. The success rate is reported to be approximately 40%. U.S. Pat. No. 5,512,275, issued to Buck, references in detail the limited commercial and clinical success of Rogain.

Other inhibitors currently being used are Saw Palmetto, Niacin, Zinc. Use of Saw Palmetto extract is described in U.S. Pat. No. 5,972,345, issued to Chizick, et al. Saw Palmetto is a weak, naturally occurring alkaloid of 5-alpha-reductase with a side effect of fluid retention and has been used in U.S. Pat. No. 5,750,108 issued to Edwards, U.S. Pat. No. 6,596,266 issued to Catalfo, et al.

Drugs may increase growth of androgen-dependent hair (hirsutism) or of all hari (hypertrichosis) hirsutism can be caused by testosterone, danazol, ACTH, metyrapone, anabolic steroids, glucocorticoids and some antiepileptic-pheytoin and carbamazepine. Hyperytichosis can be produced by cyclosporine, and minoxidil and diazoxide. Minoxidil and diazoxide open potassium channels in cell membranes leading to hyperpolarisation. The opening of potassium channels could be main mechanism of their hypertrihotic action.

### BRIEF SUMMARY OF THE INVENTION

The subject invention is directed to novel composition and method of using the same for treating male pattern baldness or androgenetic alopecia. This invention is directed to rebuilding and repairing of the hair follicles, stimulation of hair growth, and revitalization of the hair growth cycles using the synergistic effect of hyperalimentation or total parenteral nutrition, vinpocetine, niacin, proanthocyanidins, azelaic acid, folic acid, and vitamins.

The subject composition and methods offer a safe and effective means to combat androgenetic alopecia by using all naturally occurring agents. The novel composition is intended to overcome the harmful side-effects of currently available treatment for alopecia. The invention is also aimed at improving the effectiveness of treatment for alopecia.

According to the present invention, there is provided a first treatment formulation containing a combination of active ingredients comprising vinpocetine, niacin, proanthocyanidins, azelaic acid, isopropyl alcohol, propylene glycol, and water is applied to the scalp in the form of liquid suspension, lotion, gel or cream. Following the first treatment a second treatment formulation containing a combination of active ingredients comprising total parenteral nutrition or hyperalimentation, folic acid, propylene glycol, and water and is applied to the scalp in the form of liquid suspension, lotion, gel, or cream. Finally, a third treatment formulation for men containing sal palmetto, niacin, vinpocetine, folic acid, pantothenic acid, Vitamin A, Vitamin B-6, Zinc, PABA, grape seed extract, thiamine, and biotin is administered orally as a supplement. The third treatment for women contains niacin, Vitamin A, thiamine, Vitamin B-6, panothenic acid biotin, PABA, zinc, Vitamin E, Citrus bioflavonoids, calcium, riboflavin, and inositol.

### BRIEF DESCRIPTION OF THE SEVERAL VIEWS OF THE DRAWING

Not Applicable

### DETAILED DESCRIPTION OF THE INVENTION

The subject invention is directed to novel composition and method of using the same for treating male pattern baldness or androgenetic alopecia. This invention is directed to rebuilding and repairing of the hair follicles, stimulation of hair growth, and revitalization of the hair growth cycles using the synergistic effect of hyperalimentation, vinpocetine, niacin, proanthocyanidins, azelaic acid, folic acid, and vitamins.

The subject composition and methods offer a safe and effective means to combat alopecia by using all naturally occurring agents. The novel composition is intended to overcome the harmful side-effects of currently available treatment for alopecia. The invention is also aimed at improving the effectiveness of treatment for alopecia and improving the results of treatment for alopecia.

According to the present invention, there is provided a first treatment formulation containing a combination of active ingredients comprising vinpocetine, niacin, proanthocyanidins, azelaic acid, isopropyl alcohol, propylene glycol, and water is applied to the scalp in the form of liquid suspension, lotion, gel or cream. Following the first treatment a second treatment formulation containing a combination of active ingredients comprising total parenteral nutrition or hyperalimentation, folic acid, propylene glycol, and water and is applied to the scalp in the form of liquid suspension, lotion, gel, or cream. Finally, a third treatment formulation for men containing sal palmetto, niacin, vinpocetine, folic acid, pantothenic acid, Vitamin A, Vitamin B-6, Zinc, PABA, grape seed extract, thiamine, and biotin is administered orally as a supplement. The third treatment for women contains niacin, Vitamin A, thiamine, Vitamin B-6, panothenic acid biotin, PABA, zinc, Vitamin E, Citrus bioflavonoids, calcium, riboflavin, inositol

The preferred embodiment of the first treatment formulation contains the following active ingredients:

| | |
|---|---|
| Niacin | .16%-.5% |
| Proanthocyanidins | .15%-1% |
| Vinpocetine | .08%-1% |
| Azelaic Acid | 3%-5% |
| Isopropyl Alcohol | 20%-26% |
| Propylene Glycol | 8%-16% v/v |
| Water | 15%-25% v/v |

The preferred embodiment of the second treatment formulation contains the following active ingredients:

| | |
|---|---|
| Total Parenteral Nutrition (Hyperalimentation) | 7%-16% |
| Folic Acid | .01%-.16% |
| Propylene Glycol | 8%-16% V/V |
| Water | 15%-25% V/V |

The preferred embodiment of the second treatment formulation wherein the total parenteral nutrition comprising a solution of amino acids wherein the amino acid contains:

| | |
|---|---|
| L-isoleucine | .42% to 1.2% of total weight of TPN. |
| L-leucine | .54% to 1.5% |
| L-lysine | .42% to 1.2% |
| L-methionine | .23% to .66% |
| L-phenylalanine | .25% to .73% |
| L-threonine | .30% to .86% |
| L-tryptophan | .09% to .26% |
| L-serine | .24% to .7% |
| L-valine | .46% to 1.33% |
| L-tyrosine | .05% to .07% |
| L-alanine | .74% to 2.13% |
| L-arginine | .57% to 1.63% |
| L-glycine | .74% to 2.13% |
| L-proline | .5% to 1.43% |
| L-histidine | .17% to .5% |

In a more preferred embodiment of the second treatment formulation wherein the total parenteral nutrition comprising a solution of amino acids wherein the amino acid contains:

| | |
|---|---|
| L-isoleucine | .51% |
| L-leucine | .67% |
| L-lysine | .51% |
| L-methionine | .28% |
| L-phenylalanine | .31% |
| L-threonine | .37% |
| L-tryptophan | .11% |
| L-serine | .3% |
| L-valine | .57% |
| L-tyrosine | .03% |
| L-alanine | .91% |
| L-arginine | .70% |
| L-glycine | .91% |
| L-proline | .61% |
| L-histidine | .21% |

The preferred embodiment of the third treatment for men contains the following:

| | |
|---|---|
| Sal Palmetto | 100 mg to 400 mg |
| Niacin | 10 mg to 100 mg |
| Vinpocetine | 5 mg to 50 mg |
| Folic Acid | 400 mcg to 900 mcg |
| Pantothenic Acid | 20 mg to 100 mg |
| Vitamin A | 5000 u to 10,000 u |
| Vitamin B-6 | 20 mg to 100 mg |
| Zinc | 25 mg to 100 mg |
| PABA | 100 mg to 400 mg |
| Grape Seed Ext. | 20 mg to 100 mg |
| Thiamine | 10 mg to 50 mg |
| Biotin | 200 mcg to 500 mcg |

In a more preferred embodiment of the third treatment for men contains the following:

| | | |
|---|---|---|
| Sal Palmetto | .16 | gm |
| Niacin | .03 | gm |
| Vinpocetine | .01 | gm |
| Folic Acid | .0004 | gm |
| Pantothenic Acid | .03 | gm |
| Vitamin A | .003 | gm |
| Vitamin B-6 | .03 | gm |
| Zinc | .025 | gm |
| PABA | .2 | gm |
| Grape Seed Ext. | .023 | gm |
| Thiamine | .03 | gm |
| Biotin | .00025 | gm |

The preferred embodiment of the third treatment for women contains the following:

| | |
|---|---|
| Niacin | 10 mg to 100 mg |
| Vitamin A | 5000 u to 10,000 u |
| Thiamine | 10 mg to 50 mg |
| Vitamin B-6 | 20 mg to 100 mg |
| Panothenic Acid | 25 mg to 200 mg |
| Biotin | 100 mcg to 1000 mcg |
| PABA | 25 mg to 200 mg |
| Zinc | 10 mg to 100 mg |
| Vitamin E | 100 u to 1000 u |
| Citrus Bioflavonoids | 100 mg to 1000 mg |
| Calcium | 100 mg to 1000 mg |
| Riboflavin | 10 mg to 30 mg |
| Inositol | 25 mg to 250 mg |

In a more preferred embodiment of the third treatment for women contains the following:

| | | |
|---|---|---|
| Niacin | .025 | gm |
| Vitamin A | .003 | gm |
| Thiamine | .025 | gm |
| Vitamin B-6 | .025 | gm |
| Panothenic Acid | .025 | gm |
| Biotin | .0001 | gm |
| PABA | .025 | gm |
| Zinc | .015 | gm |
| Vitamin E | .134 | gm |
| Citrus Bioflavonoids | .1 | gm |
| Calcium | .2 | gm |
| Riboflavin | .025 | gm |
| Inositol | .025 | gm |

In another embodiment of the invention the first and second treatment formulation composition is in the form of liquid suspension, gel, lotion, foam, styling mousse, styling hair tonic and styling hair spray.

Another embodiment of the invention is the combination of the said first treatment formulation and said second treatment formulation for treatment of male pattern baldness.

In another embodiment of the invention the third treatment formulation is administered orally as a hair nutritional supplement.

According to another embodiment of the subject invention is directed to a method for using the first treatment, second treatment, and third treatment. The method comprises: applying 1 mL to 2 mL of said first treatment solution twice a day once in the morning and once in the evening to the frontal and crown regions of the scalp; applying 1 mL to 2mL of said second treatment is applied twice a day once in the morning and once in the evening five minutes after applying the said first treatment solution to the frontal and crown of the scalp; applying first treatment solution and second treatment solution continuously as a hair revitalizing supplement; and orally administering said third treatment once a day as a hair nutritional supplement.

The effectiveness and the novelty of the present invention is based on two major components. The first is the amino acid solution that is based on the synergistic effect of hyperalimentation or Total Parenteral Nutrition (TPN), vinpocetine, niacin, proanthocyanidins, azelaic acid, and folic acid.

Diseases resulting from deficiencies of specific nutrients in the diet are well recognized and excepted in the underdeveloped areas of the world, problems of nutritional homeostatic in hospitalized patients are generally a secondary consequence rather than a cause of diseases. The intake of nutrients may be interfered within a variety of inflammatory, septic, or malignant disorders, as a result of anorexia or vomiting, in association with primary disturbances of the gastrointestinal tract and in the postoperative state.

Total Parenteral Nutrition has been applied in a variety of clinical conditions. The following disorders are likely to benefit from this form of therapy: (1) Gastrointestinal disorders, particularly chronic inflammatory bowel disease (regional enteritis, ulcerative colitis), complicated by fistula formations; the short bowel syndrome after extensive intestinal resection and congenital lesions of the gastrointestinal tract amenable to surgical repair, (2) Extensive body burns and traumatic injuries in which caloric requirements are increased.

A variety of hypertonic glucose and amino acid mixtures are currently available in commercial form. Glucose concentrations range from generally 20% to 50%. Amino acid formulations for TPN range from 3.5% to 10%. The concentration of amino acids in the present invention is approximately 4.2%, based on the commercial formulations used and is sold in commerce under the name Aminosyn by Abbot Laboratories Inc.

The present invention incorporates essential and nonessential amino acids. Based on the crystalline amino acids, solution used in hyperalimentations, these agents promote protein synthesis and increase follicle activity at the hair matrix cell level. The amino acids associated in TPN solutions increase the synthesis of stromelysis, which act on the papilla cells and accelerate their growth. In terms of size (molecular weight) and variety of constituents units, proteins are considerably more complex than either carbohydrates or lipids. When boiled eighteen to twenty-four hours with moderate concentration HCl, proteins are hydrolyzed into their constituents units, which are called amino acids. By various physical methods all of the amino acids can be isolated and purified in fairly good yields, except tryptophan, which condenses into acidic solution with aldehydes, such as glucose and the decomposition products of some of the amino acids to form a black, insoluble tar. Some 18 to 20 different amino acids can be isolated from most proteins; in most cases the number of molecules (or residues, as the constituents units of protein are called) of each amino acid is large enough to make the total molecular weight of the typical protein, at least 10,000. The molecular weight of some proteins is as large as 1,000,000.

Although more than 30 amino acids have been isolated and identified as constituents of various proteins, only 20 are found in the majority of common proteins. Although few are present in all proteins and conversely, few proteins contain every amino acid, all are present in sufficiently high concentrations in some proteins, to consider them all of a general biochemical significance.

Vinpocetine, a derivative of Vincamine, is an alkaloid of the common periwinkle plant, Vinca Minor. Vinpocetine has been used to help improve memory, learning ability, insomnia, hearing, eyesight, effects of menopause, increase male and female libido, and increase tolerance to damage caused by hypoxia. Similar to Minoxidil, it dilates arteries and capillaries in the head area. Chemically 14-ethoxycarbonyl-3 alpha, 16 alpha-ethyl-14, 15-ebumamine (C22 H26 N2 O2) of vinpocetine increases blood flow, which increases cellular ATP and regulates sodium and potassium channels. Chemically 6-1-piperidinyl-2-4 pyrimidinediamine 2-oxide (C9 H15 N5 0) of Minoxidil shares some of the common structural components. Originally founded as an angina/hypertensive agent, this drug caused too many side effects when used orally. This agent was known to cause hypertrichosis, which occurred then was exploited in treating androgenetic alopecia. Both agents can be used in exchange. Vinpocetine has not been associated with any of the side effects of Minoxidil. The present invention incorporates 0.08% concentration of vinpocetine, while Minoxidil containing products range from 2% to 5%. Also from a chemical structure study the structure of vinpocetine is similar to Propecia marketed by the company Merck. Vinpocetine blocks 5-alpha-reductase as well as Propecia without the side effect of sexual disfunction.

Niacin is a weak vasodilator to increase blood circulation. Niacin is commonly used for vitamin B1 deficiencies. The present invention uses Niacin as a topical vasodilator. Niacin is also used in skin abnormalities as described in US Patent 20040081672.

WO 6/00561 and U.S. Pat. No. 6,562,804 describe hair-growing agents comprising proanthocyanidin. It has been proposed that proanthocyanidins possess growth-promoting activity toward murine hair epithelial cells in vitro and stimulating anagen induction in hair cycle progression in vivo.

Azelaic acid has been well established as dihydrotestosterone (DHT) blocker to prevent hair loss. U.S. Pat. No. 6,358,541 uses azelaic acid as low irritability penetration enhancer.

Folic acid, also known as pteroylglutamic acid is incorporated in the present invention due to its positive effect on growth of cellular energy, adenotriphosphate (ATP).

The preparation of the present invention would result in two separate treatment solutions. The first treatment solution is considered to be the “stimulant” and the second treatment acts the “nutrient.” The first treatment is prepared by grinding azelaic acid into fine powder then vinpocetine is added to the mortar and also ground to a fine powder. Vinpocetine is only soluable in DMSO, 100% ethanol or acetone and therefore suspension will be the result. It is slightly soluable in Isopropyl alcohol and added to the mortar to act as a carrier. In a separate mortar niacin and proanthocyanidins, obtained from grape seeds, are weighed and mixed in sterile water. The two products are then combined and mixed together with propylene glycol.

Sample:

3 grams of azelaic acid was ground up into a powder from “hairs” of this dicarboxylic acid. Soluble in hexane,toluene,ethanol and acetone, insoluble in propylene glycol, first treatment resulted in a suspension. Next, 50 mg of vinpocetine is added to the mortar and also ground to a powder. The use of 10 mg tablets of vinpocetine can be used if the raw material in not available. Vinpocetine is only soluble in DMSO, 100% ethanol or acetone. At this point 26 cc (or ml) of isopropyl alcohol is added to the mortar and mixed to form the first suspension. Next, in a separate mortar 100 mg of niacin and 100 mg of grape seed (that contain 95 mg of proanthrocyanidins)) are weighed and mixed together. These two powders are soluble in water which 5 ml of sterile water is added and stirred. At the point the two products can be combines with the final addition of 12 ml of propylene glycol. This agent is used as a solvent for many topical pharmaceutical products. The final volume of 60 ml can be achieved by the “qs” or “adding up to” the 60 ml mark with sterile water. Additional fragrances can also be added.

The second treatment is prepared by extracting amino acids from hyperalimentation or TPN, glucose free. Crystalline solution of Aminosyn 10% was used to extract the amino acids. Next, folic acid was added to the amino acids along with an “alcohol free toner” by Neutrogena in order to neutralize the pungent odor of the TPN solution. Finally, water and propylene glycol are added.

Sample:

The nutrients of second treatment contain amino acids and folic acid as indicated by the formula along with the amounts. The amino acids used in the sample was extracted directly from the crystalline solution Aminosyn 10%. 43 mls of Aminosyn 10% were used along with Folic Acid (5 mg/2 cc) 2 cc injection to supply the topical nutrients. To this 15 ml of “alcohol Free Toner” by Neutrogena was used to neutralize the pungent odor of the crystalline TPN solution. Alternatively this bottle can be made by weighing out all the appropriate amounts of amino acids and folic acid and dissolving them in 40 ml of sterile water. To this 10 ml of propylene glycol will be added and “qs” to 60 ml with sterile water.

Care has been taken to separate first treatment, the stimulant agent, from the second treatment, the nutrients, since stability and incompatibilities are not yet known to insure a shelf life of at least 24 months. Combining the two treatments evidenced no such instability.

The composition of the first treatment and second treatment should be administered topically to the frontal to the crown of the scalp. Prior to the application the scalp should be prepared by using a mild shampoo. Following the topical administration the third treatment should be orally administered. During the use of the treatment hair products such as hair spray, styling tonics, gels, foams, mousse, hair coloring, and permanence should not be used.

For improved fragrance and longer shelf life of the first and second treatment preservatives and stabilizers can be added so that the treatments maintain its integrity.

The first treatment and second treatment should be used twice a day once in the morning and once in the evening. 1 ml-2 ml of first treatment should be applied to a clean scalp followed by 1 ml-2 ml of second treatment five after the first treatment. Lastly, the third treatment in the form of tablets should be orally administered once a day.

Many of the gray hairs have turned into their original color, but not in all cases probably due to tyrosine, that is contained in the formulation. It has been observed that as long as the hair follicles are somewhat functioning, rejuvenation will take place.

What is claimed:

1. A composition and method for revitalizing hair growth, rebuilding and restoring the natural integrity of the hair follicles wherein a first treatment formulation containing a combination of active ingredients comprising vinpocetine, niacin, proanthocyanidins, azelaic acid, isopropyl alcohol, propylene glycol, and water, a second treatment formulation containing a combination of active ingredients comprising total parenteral nutrition or hyperalimentation, folic acid, propylene glycol, and water, a third treatment formulation for men containing sal palmetto, niacin, vinpocetine, folic acid, pantothenic acid, Vitamin A, Vitamin B-6, zinc, para-amino benzoic acid, grape seed extract, thiamine, and biotin, and a third treatment for women containing niacin, vitamin A, thiamine, vitamin B-6, panothenic acid, biotin, para-amino benzoic acid, zinc, vitamin E, citrus bioflavonoids, calcium, riboflavin, and inositol.

2. The composition of claim 1 wherein said first treatment formulation is combined with said second treatment formulation.

3. The first treatment formulation composition as defined in claim 1, comprising approximately the following active ingredients:

| | |
|---|---|
| Niacin | .16%-.5% |
| Proanthocyanidin | .15%-1% |
| Vinpocetine | .08%-1% |
| Azelaic Acid | 3%-5% |
| Isopropyl Alcohol | 20%-26% |
| Propylene Glycol | 8%-16% v/v |
| Water | 15%-25% v/v |

4. The first treatment formulation composition as defined in claim 1 wherein the composition further contains for application to the scalp selected from the group consisting of liquid suspension, gel, lotion, foam, styling mousse, styling hair tonic and styling hair spray.

5. The second treatment formulation composition as defined in claim 1, comprising, approximately the following active ingredients:

| | |
|---|---|
| Total Parenteral Nutrition | 7%-16% |
| Folic Acid | .01%-.16% |
| Propylene Glycol | 8%-16% V/V |
| Water | 15%-25% V/V |

6. The second treatment formulation composition as defined in claim 5 wherein total parenteral nutrition comprising a solution of amino acids wherein the amino acid content consists essentially of:

| | |
|---|---|
| L-isoleucine | .42% to 1.2% of total parenteral nutrition |
| L-leucine | .54% to 1.5% of total parenteral nutrition |
| L-lysine | .42% to 1.2% of total parenteral nutrition |
| L-methionine | .23% to .66% of total parenteral nutrition |
| L-phenylalanine | .25% to .73% of total parenteral nutrition |
| L-threonine | .30% to .86% of total parenteral nutrition |
| L-tryptophan | .09% to .26% of total parenteral nutrition |
| L-serine | .24% to .7% of total parenteral nutrition |
| L-valine | .46% to 1.33% of total parenteral nutrition |
| L-tyrosine | .05% to .07% of total parenteral nutrition |
| L-alanine | .74% to 2.13% of total parenteral nutrition |
| L-arginine | .57% to 1.63% of total parenteral nutrition |
| L-glycine | .74% to 2.13% of total parenteral nutrition |
| L-proline | .5% to 1.43% of total parenteral nutrition |
| L-histidine | .17% to .50% of total parenteral nutrition |

7. The second treatment formulation composition as defined in claim 6 wherein total parenteral nutrition comprising a solution of amino acids wherein the amino acid content consists essentially of:

| | |
|---|---|
| L-isoleucine | .51% |
| L-leucine | .67% |
| L-lysine | .51% |
| L-methionine | .28% |
| L-phenylalanine | .31% |
| L-threonine | .37% |
| L-tryptophan | .11% |
| L-serine | .30% |
| L-valine | .57% |
| L-tyrosine | .03% |
| L-alanine | .91% |
| L-arginine | .70% |
| L-glycine | .91% |
| L-proline | .61% |
| L-histidine | .21% |

8. The second treatment formulation composition as defined in claim 1 wherein the composition further contains for application to the scalp selected from the group consisting of liquid, gel, lotion, foam, styling mousse, styling hair tonic, styling hair spray, hair permanence, and hair coloring.

9. The third treatment formulation composition for men as defined in claim 1 wherein the composition comprising of sal palmetto, niacin, vinpocetine, folic acid, pantothenic acid, Vitamin A, Vitamin B-6, Zinc, PABA, grape seed extract, thiamine, and biotin.

10. The third treatment formulation composition for men as defined in claim 9 wherein the composition consists essentially of:

| | |
|---|---|
| Sal Palmetto | 100 mg to 400 mg |
| Niacin | 10 mg to 100 mg |
| Vinpocetine | 5 mg to 50 mg |
| Folic Acid | 400 mcg to 900 mcg |
| Pantothenic Acid | 20 mg to 100 mg |
| Vitamin A | 5000 u to 10,000 u |
| Vitamin B-6 | 20 mg to 100 mg |
| Zinc | 25 mg to 100 mg |
| PABA | 100 mg to 400 mg |
| Grape Seed Ext. | 20 mg to 100 mg |
| Thiamine | 10 mg to 50 mg |
| Biotin | 200 mcg to 500 mcg |

11. The third treatment formulation composition for men as defined in claim 10 wherein the composition consists essentially of:

| | | |
|---|---|---|
| Sal Palmetto | .16 | gm |
| Niacin | .03 | gm |
| Vinpocetine | .01 | gm |
| Folic Acid | .0004 | gm |
| Pantothenic Acid | .03 | gm |
| Vitamin A | .003 | gm |
| Vitamin B-6 | .03 | gm |
| Zinc | .025 | gm |
| PABA | .2 | gm |
| Grape Seed Ext. | .023 | gm |
| Thiamine | .03 | gm |
| Biotin | .00025 | gm |

12. The third treatment formulation composition for women as defined in claim 1 wherein the composition comprising of niacin, vitamin A, thiamine, vitamin B-6, pantothenic acid, biotin, PABA, zinc, vitamin e, citrus bioflavonoids, calcium, riboflavin, and inositol.

13. The third treatment formulation composition for women as defined in claim 12 wherein the composition consists essentially of:

| | |
|---|---|
| Niacin | 10 mg to 100 mg |
| Vitamin A | 5000 u to 10,000 u |
| Thiamine | 10 mg to 50 mg |
| Vitamin B-6 | 20 mg to 100 mg |
| Panothenic Acid | 25 mg to 200 mg |
| Biotin | 100 mcg to 1000 mcg |
| PABA | 25 mg to 200 mg |
| Zinc | 10 mg to 100 mg |
| Vitamin E | 100 u to 1000 u |
| Citrus Bioflavonoids | 100 mg to 1000 mg |
| Calcium | 100 mg to 1000 mg |
| Riboflavin | 10 mg to 30 mg |
| Inositol | 25 mg to 250 mg |

14. The third treatment formulation composition for women as defined in claim 13 wherein the composition consists essentially of:

| | | |
|---|---|---|
| Niacin | .025 | gm |
| Vitamin A | .003 | gm |
| Thiamine | .025 | gm |
| Vitamin B-6 | .025 | gm |
| Panothenic Acid | .025 | gm |
| Biotin | .0001 | gm |
| PABA | .025 | gm |
| Zinc | .015 | gm |
| Vitamin E | .134 | gm |
| Citrus Bioflavonoids | .1 | gm |
| Calcium | .2 | gm |
| Riboflavin | .025 | gm |
| Inositol | .025 | gm |

15. The method of claim 1 wherein 1 mL to 2 mL of said first treatment solution is applied twice a day once in the morning and once in the evening to the frontal and crown regions of the scalp.

16. The method of claim 1 wherein 1 mL to 2 mL of said second treatment is applied twice a day once in the morning and once in the evening five minutes after applying the said first treatment solution to the frontal and crown of the scalp.

17. The method of claim 1, wherein the third treatment formulation is administered orally once a day continuously as a daily nutritional supplemental.
